(19) United States
(12) Patent Application Publication
(10) Pub. No.: US 2014/0114332 A1
(43) Pub. Date: Apr. 24, 2014

(54) SURGICAL CLIP

(71) Applicant: Theodor Lutze, Balgheim (DE)

(72) Inventor: Theodor Lutze, Balgheim (DE)

(73) Assignee: Aesculap AG, Tuttlingen (DE)

(21) Appl. No.: 14/009,568

(22) PCT Filed: Mar. 21, 2012
(86) PCT No.: PCT/EP2012/055021

§ 371 Date: Dec. 17, 2013

(30) Foreign Application Priority Data
Apr. 5, 2011 (DE) 10 2011 001 798.4

(51) Int. Cl. A61B 17/08

(52) U.S. Cl. CPC A61B 17/083
USPC 606/151

(57) ABSTRACT

A surgical clip includes two clip branches each consisting of a preferably straightly extending (bar-shaped) clamping portion and an actuating portion as well as comprising a flexural spring arrangement by which the two clip branches are coupled in one piece. The flexural spring arrangement is arranged and formed so that it bulges between the clip branches along the actuating portions toward the clamping portions. The flexural spring arrangement is composed by a number of serially coupled individual spring arms.

### RELATED APPLICATIONS

This application is the U.S. National Phase of International Application No. PCT/EP2012/055021, filed Mar. 21, 2012, which claims the benefit of priority of German Patent Application No. DE 10 2011 001 798.4, filed Apr. 5, 2011. The contents of both applications are incorporated by reference herein for all purposes.

### FIELD

The present invention relates to a surgical clip.

### BACKGROUND

Surgical clips (or else referred to as tissue clips) are medical implants which are mostly used temporarily for closing tissue perforations, for example, or for treating aneurysms as vascular clips. For this, a plurality of clip shapes starting from jaw-like clips in which the clamping or clipping branches are shaped similarly to upper and lower jaws as two opposed longitudinally bent rows of teeth that are coupled to each other via hinges at their respective two longitudinal ends up to row-like (straight) clips which, comparably to a conventional pair of forceps, include two opposed substantially straight clamping rails that may be equipped with teeth or a corrugation and that are coupled hinge-like to each other only at one respective longitudinal end.

However, all known clip shapes have in common at least one spring arrangement applying a clamping force to the clip branches. Said at least one spring arrangement can be in the form of a separate component inserted in the clip so as to bias the clip branches against each other, or it is integrated (in one piece) in the clip. In the latter case the spring arrangement substantially constitutes the hinge or hinges to which the two clip branches are pivotally coupled.

From the state of the art a surgical clip of the species having straight clip branches and a one-sided spring arrangement is known, as it is published, for example, in DE 20 2010 008 512 U1.

The clamping or closing force is generated in this case by a leg spring in the form of either a circular spring or a rectangular spring. Concretely speaking, such clip consists of two clip branches loosely intersecting at their respective central portions (i.e. without any mechanical connection such as a hinge pin) so as to form two clamping portions adjacent to each other in parallel in the closing position and two actuating legs or actuating portions spaced apart at said position. The free ends of the actuating portions are interconnected via the afore-mentioned leg spring which is formed either integrally (of one single piece) with the clip branches or as a separate component and is subsequently connected (soldered, welded, assembled etc.) with the clip branches to form one part. The leg spring can exhibit either a half winding or a 1.5 fold or even a 2.5 fold winding.

For the purpose of actuation the surgical clip is compressed at its actuating portions, thereby the clamping portions being spaced apart due to the intersecting alignment of the clip branches. At the same time, the leg spring is biased more strongly. When the actuating portions are released, the leg spring causes the actuating portions to be forced apart until the clamping portions are pressed against each other.

Although the afore-described design ensures a sufficiently high pressing force between the two clamping portions and the leg spring is loaded little due to a merely low degree of elastic deformation and therefore is very durable, several drawbacks are resulting.

The use of the leg spring requires a comparatively expensive and difficult manufacturing process for coiling the spring, which has to take place without fissuring of the spring material. In this respect, it is also possible that the leg spring is overwound, thereby the pressing force between the clamping portions being reduced. On the whole, the manufacture of the leg spring therefore requires a high degree of precision to produce instruments the characteristics of which (pressing force, service life, operating safety etc.) are within a narrow tolerance range. Consequently, the scrap rate is correspondingly high. All of the afore-mentioned and further drawbacks of the known surgical clip finally result in a comparatively high price for the surgical clip.

From another state of the art according to DE 20 2010 008 714 U1 a surgical clip is known comprising two clip branches that extend in parallel to each other without intersecting and that are connected in their respective central portions via a web-shaped flexural spring. The clip branches are consequently lengthened beyond the flexural spring in the longitudinal direction thereof and in this way form two clamping portions on the one side of the flexural spring and two actuating legs/portions on the other side of the flexural spring, similarly to the principle of a generally known clothes peg. If thus the two actuating portions are pressed against each other on the one side of the web-shaped flexural spring, on the other side of the flexural spring the two clamping portions are moving away from each other, and vice versa.

Said surgical clip is made of one single part, for example as punched or laser-cut part, and thus is relatively inexpensive in terms of manufacture. In contrast to the afore-described clip, the punching or laser cutting process allows to carry out the manufacture substantially automatically and thus in an extremely precise manner. The courses of bending and spring force are easily reproducible within a comparatively narrow tolerance even in large quantities. Thus, a high quality level can be easily obtained. This solution also exhibits drawbacks though.

Although the flexural spring has an open annular shape bulging toward the actuating portions, the distance/degree of deformation of the flexural spring during typical use is comparatively high so that the spring can rapidly fatigue. In addition, the bending stress compared to the leg spring is by far higher, thereby spring fractures being more frequently possible. This is finally resulting in a lower operating safety and service life vis-à-vis the leg spring.

Ultimately, the state of the art also has endeavored to arrange the annular or U-shaped flexural spring at the respective outer ends of the clip branches so that clamping and actuating portions of each clip branch are located on the same side of the spring and thus have the same direction of motion when the clip is actuated (and not an opposed one as in the afore-mentioned state of the art). In this case the clip is spread by pulling the actuating portions apart and not by compressing them as in the state of the art mentioned in the beginning. This results in the clip becoming unhandy/non-ergonomic with its scope of application being definitely reduced. Alternatively, it is basically possible to intersect the branches so that compressing of the actuating portions causes the clamping portions to be opened. Also in this case, however, at least the biasing force of the spring has turned out to be comparatively low so that the material strength of the spring had to be increased. Therefore, in total the clip is dimensioned to be so large that it is only suited for particular purposes.

### SUMMARY

In view of these problems, it is the object of the present invention to provide an inexpensive surgical clip having improved characteristics. It is one objective for the clip to exhibit high operating safety and to be adapted to most versatile use. It is another objective for the clip to offer maximum closing force and opening width with an as small design as possible. In addition, the clip should preferably exhibit an (integrated) protective mechanism against mechanical overload as a result of improper handling, for example.

This object is achieved by a surgical clip in accordance with the invention. In one embodiment, a surgical clip includes two clip branches each consisting of a clamping portion and an actuating portion as well as comprising a flexural spring arrangement by which the two clip branches are coupled to each other in one piece. The flexural spring arrangement bulges at least in partial areas between the clip branches along the actuating portions toward the clamping portions.

In one advantageous configuration, the flexural spring arrangement is composed of a plurality of serially coupled spring arms.

In another advantageous configuration, the flexural spring arrangement forms a turning preferably having a U, C, V or W shaped outline in its bulging partial area.

In another advantageous configuration, in the area of the bulge, the flexural spring arrangement forms at least two spring arms connected in one piece to the axial free ends of the actuating portions of the two clip branches.

In another advantageous configuration, the flexural spring arrangement includes a connecting web for interconnecting the at least two spring arms at their respective one end facing the clamping portions, wherein the connecting web forms a stop for the actuating portions to delimit the actuating path thereof.

In another advantageous configuration, the connecting web projects beyond the connecting points including the spring arms in the direction of the two actuating portions of the clip branches so as to form an actuation limit stop.

In another advantageous configuration, the surgical clip has a straight branch design, with each of the two opposing clamping portions thereof exhibiting a straightly extending clamping bar preferably equipped with a toothing or corrugation.

In another advantageous configuration, the two rear ends of the two clamping bars facing each of the actuating portions are lengthened into two intermediate pieces which in the construction position of the clip extend away from each other at an obtuse angle with the respective clamping bars.

In another advantageous configuration, the two opposed intermediate pieces are converted, at an obtuse angle, into the two actuating portions which approach each other in the direction of their rear free ends.

In another advantageous configuration, each of the two actuating portions forms an actuating bar which is spring-elastic over its entire or partial length so as to serve as further serially arranged spring arms of the flexural spring arrangement.

In another advantageous configuration, the surgical clip is a sheet component which is preferably manufactured by punching or laser-cutting.

In another advantageous configuration, the flexural spring arrangement is made of spring steel separately from the clip branches and is connected to the two clip branches in one piece preferably by soldering or welding.

In another advantageous configuration, the surgical clip is manufactured in one piece of one single material.

In another advantageous configuration, the surgical clip branches extend while being loosely intersected such that the clamping portions and the actuating portions are functionally arranged on the one common side of the flexural spring arrangement, wherein movement of the actuating portions toward each other causes the clamping portions to move apart.

On principle, a basic idea of the invention consists in making use of a flexural spring or flexural spring arrangement the overall spring length of which is extended to a maximum, for example by plastic/permanent folding or bending (plastic deformation during the manufacturing process) of the flexural spring or by (preferably fold-like) serial arrangement of individual springs of a flexural spring arrangement. Another or additional basic idea of the invention provides that the flexural spring or the flexural spring arrangement extends inwardly, i.e. in the direction of the clamping portions at least in partial areas starting from the actuating portions of the clip branches.

In this way, the bending degree of the flexural spring/flexural spring arrangement and of the individual springs, respectively, can be reduced and thus the operating safety can be increased. Further, due to the use of flexural springs a sufficiently high biasing force can be obtained without the surgical clip having to be structurally enlarged/ lengthened.

The surgical clip according to a special aspect of the invention consequently comprises two clip branches, each consisting at least of a clamping portion and an actuating portion, as well as a flexural spring arrangement by which the two clip branches, especially the actuating portions thereof, are coupled in one piece. The flexural spring arrangement forms at least functionally and/or structurally two spring legs or arms which are serially interconnected so that (in a central portion) the flexural spring arrangement forms sort of a turning (approximately 180°). Said at least two spring arms are furthermore aligned/arranged at the clip so that the turn faces toward the clamping portions. In other words, according to the invention a flexural spring or flexural spring arrangement is resulting which forms at least one turning or indentation extending along the clip branches toward the clamping portions.

By this technical measure the bending distance of the spring (i.e. the effective total length of the spring arm) is enlarged and thus the degree of deformation thereof or of the individual spring arms is reduced. The total dimension of the clip is not or only slightly enlarged (lengthened), however, as the turning does not extend in a direction away from the clamping portions, as this is the case in the state of the art, but in the opposite direction. In this way, a great biasing force can be achieved with a low risk of fracture and a compact design so that also the scope of application of the clip is extended by reason of this compact design. Since, moreover, the clip utilizes a flexural spring (substantially two-dimensional) rather than a spiral or leg spring having complex (three-dimensional) geometry, it can be manufactured in a simple fashion at low cost.

It is advantageous when the functionally and/or structurally at least two serially disposed spring arms of the flexural spring or flexural spring arrangement form a U, C, W or V-shaped outline which consequently constitutes the basic shape of the turning. Such simple geometrical basic shape can be manufactured in an easy and precise fashion and thus entails a further reduction of costs. It is noted in this context that also other turning shapes such as a π shape or Ω shape are possible or the afore-mentioned basic shapes can be superimposed by further sub-shapes such as corrugated or zigzag-shaped spring arms, thereby allowing the effective spring length per spring arm to be even further enlarged.

In accordance with another or additional aspect of the invention, the flexural spring/flexural spring arrangement of the surgical clip is integrally connected to the axial free ends of the actuating portions of the two branches via its spring legs/arms. Thus an especially favorable introduction of bending force to the flexural spring is achieved (without any additional lever action).

It is noted in this context that the actuating portion of each clip branch can be rigid or spring-elastic at least over a partial distance (or completely) and thus can form part of the flexural spring arrangement. In other words, it is possible to functionally/structurally equip the flexural spring/flexural spring arrangement with two spring arms (as afore-described) such that the free ends of the spring arms extend in a direction away from the clamping portions of the clip branches. In this case the free ends of said two spring arms are connected to the rear end areas/ends of the actuating portions. If the actuating portions are manually compressed, said two spring arms experience an elastic bending deformation along their arm length, i.e. the turning is elastically spread. The degree of deformation of the flexural spring arrangement is thus exclusively resulting from the total length of said two spring arms in the case of comparatively rigid actuating portions.

It is also possible, however, to design the actuating portions so as to be elastic at least over a partial length or completely, i.e. to deliberately assign them to elastic deformation in the typical case of actuation. In this case, there is resulting a flexural spring or a flexural spring arrangement including the two afore-mentioned spring arms to which a further flexural spring arm is serially connected at the respective end side so that the outline is constituted by a W-shaped flexural spring arrangement (including 4 individual springs). The alignment of the flexural spring arrangement remains unchanged. I.e. the two outer spring arms now simultaneously form the actuating portions or at least a partial distance thereof, with the free (rear) ends thereof being serially coupled to the spring arms defining the turning. In this way the degree of deformation of each individual spring arm can be further reduced.

Another or additional aspect of the invention provides that at the outermost area of the turning the flexural spring forms a connecting web which interconnects the at least two spring arms at the respective end thereof facing the clamping portions, the connecting web projecting from the connecting points including the spring arms in the direction of the two actuating portions of the clip branches. In this way, the spring outline approximately takes on a π shape as already mentioned by way of example in the foregoing. This constructional measure helps to reduce stress peaks in the transition zones of the spring legs/arms and thus to further reduce the risk of fracture. Optionally, this measure can be assisted by forming a rounding at the connecting web in the transition zone between the two spring arms preventing or reducing, respectively, a notch effect.

In accordance with another or additional aspect of the invention, the clip of the straight branch design preferably is an aneurysm clip the two opposed clamping portions of which have at least in portions a straightly extending bar preferably equipped with a toothing or corrugation.

It is of advantage in this context when the two rear ends of the two (straight) bars facing each of the actuating portions are lengthened to form two intermediate pieces moving towards each other at an angle or at an arc with the respective (straight) bars and loosely intersecting (without a pivot/joint connection). In this manner, as is also known from the state of the art, a kinematic reversal of the courses of motions between the actuating portion and the clamping portion can be obtained. That is to say, when the two actuating portions are compressed, the clip branches spread in the area of the clamping portions, and vice versa.

It is moreover advantageous when the two opposed intermediate pieces are converted, each at an obtuse angle or in reversing the arc, into the two actuating portions of the clip branches which are now approaching in the direction of their rear free ends. Thus an ergonomic shape is obtained in which the actuating portions can be seized more easily by one hand. Moreover, hereby a distance into which the at least one turning or indentation of the flexural spring arrangement can extend is provided between the actuating portions.

Another or additional aspect of the invention provides that the connecting web forming at the end of the turning and especially the end-side extensions thereof in the direction of the two actuating portions form or include respective stops which, when the clip is completely open, i.e. when the actuating portions are compressed, are adjacent thereto and thus prevent the clip from further opening. This constitutes a structurally simple measure against overstretching the flexural spring and thus a protection against material fatigue or spring fracture.

The surgical clip according to the invention in accordance with another or additional aspect is a sheet component which is machined preferably by punching or laser-cutting. The flexural spring can moreover be manufactured of separate spring steel and can be connected in one piece to the two clip branches preferably by soldering or welding. Alternatively, the surgical clip can be manufactured in one piece of one single material and can be heat-treated e.g. in portions in accordance with its function.

Ultimately, it is provided according to another or additional aspect of the invention that clip branches are loosely intersecting, wherein the clamping and actuating portions are functionally arranged on the same side of the flexural spring so that moving toward each other (compressing) of the actuating portions causes the clamping portions to move apart from each other (to open). This design can be manufactured in a simple and thus inexpensive fashion, as only few subsequent reshaping steps of the punched, cut, laser-cut or water jet-cut sheet are necessary.

### BRIEF DESCRIPTION OF THE DRAWING FIGURES

Hereinafter the invention will be explained in detail by way of a preferred embodiment with reference to the accompanying figures.

FIG. 1 shows a surgical clip (or tissue clip) according to the invention of the straight branch design for instance for use as an aneurysm clip in the construction position and thus stress-free,

FIG. 2 shows the surgical clip according to FIG. 1 in a (tensioned) clamping position (hereinafter referred to as idle state) and

FIG. 3 shows the surgical clip according to FIG. 2 in a (further tensioned) open position (hereinafter referred to as supply state).

### DETAILED DESCRIPTION

The surgical clip according to a preferred embodiment of the invention includes two clip branches 1 each at least consisting of a clamping portion 2 and an actuating portion 4 as well as including a flexural spring or flexural spring arrangement 6 connecting the clip branches which indents or bulges at least over a partial area along the actuating portions 4 in the direction of the clamping portion 2 preferably in U or V shape.

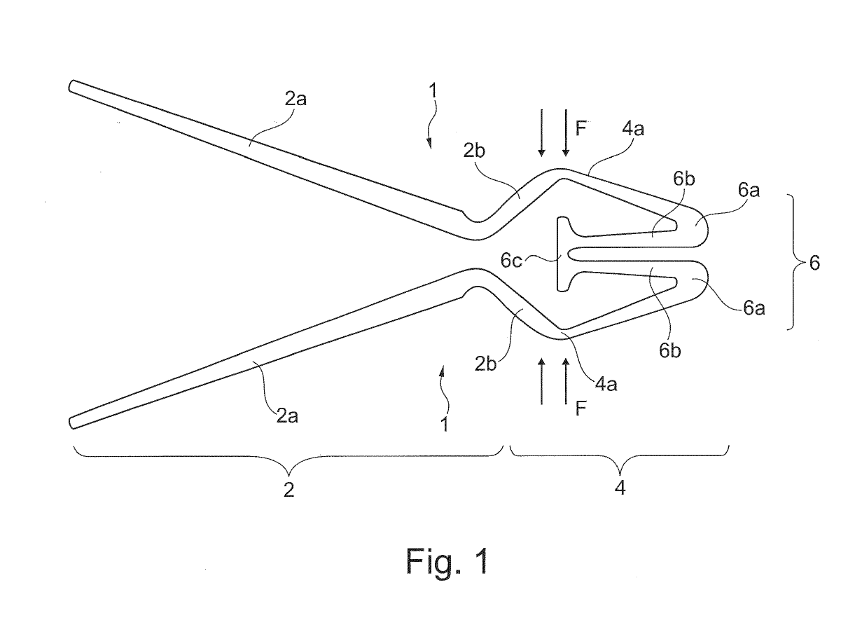

The clamping portion 2 of each clip branch 1 according to FIG. 1 comprises a substantially straight elongate clamping bar 2a which is equipped with a toothing or provided with a corrugation at a side facing the other clamping bar 2a (not shown in detail). However, it can also be formed to have a rough surface or to be simply smooth (unmachined surface). The front free ends of each clamping bar 2a are rounded or provided with a protective cap to prevent injuries. Moreover, each clamping bar 2a can be provided or designed with a reinforcing strip preferably at a side facing away from the other clamping bar 2a which reinforcing strip ensures higher flexural stiffness of each clamping bar 2a at least in the clamping or active direction.

The rear end of each clamping bar 2a is lengthened into a lengthening or intermediate piece 2b of the clamping portion 2 which according to FIG. 1 moves away from the respective opposite clip branch 1 at an (obtuse) angle or an arc, thereby a continuously widening gap or space being formed between the two clip branches 1 in the axial area of the intermediate pieces 2b. The actuating portion 4 presently in the form of an actuating bar 4a is axially connected to the rear end of each intermediate piece 2b while forming an (obtuse) angle or continuing the arc such that the two opposed actuating bars 4a gradually approach in the extension direction thereof and thus reduce the gap/space.

In the shown embodiment the actuating bars 4a are formed to be substantially straight and non-profiled. However, they can also have a longitudinal shape adapted to the human hand (finger) (e.g. slightly corrugated/curved) or an actuating key (not shown) can be put (soldered) onto the actuating bars 4a.

According to the invention, the actuating bars 4a are resilient. I.e. the actuating bars 4a are provided for elastically deforming (bending) upon actuation of the surgical clip. Thus they constitute part of the flexural spring arrangement 6. As an alternative hereto, they can also be formed to be rigid over the entire length thereof (hence not pertaining to the flexural spring arrangement) or to be elastic only over a partial length, preferably over the rear bar area, and thus partly pertaining to the flexural spring arrangement.

Each of the free rear ends of the actuating bars 4a is converted into the flexural spring or a further part (partial area) of the flexural spring arrangement while forming a rounded edging 6a.

It is pointed out in this context that the surgical clip according to the invention is shown in FIG. 1 in the construction position, i.e. in a position at the time of its manufacture in a non-tensioned state. In this state the two clip branches 1 extend substantially in parallel to each other (without intersecting), wherein the flexural spring arrangement 6 is provided at the end of the actuating portions 4. I.e. the clamping and actuating portions 2, 4 are functionally provided on one side of the flexural spring arrangement 6.

According to FIG. 1, each rounded edging 6a is (integrally) connected to a (further) spring arm/spring leg 6b of the flexural spring arrangement 6 which in the shown construction position extend substantially in parallel to each other within the gap/space defined by the opposed intermediate pieces 2b and actuating bars 4a of both clip branches 1 along or axially back in the direction of the clamping portions 2a. In the present case the two spring arms 6b extend up to the height of the transitions from the two intermediate pieces 2b to the actuating bars 4a and are interconnected there by a connecting or cross web 6c. Hereby in this partial portion basically the outline of an (inwardly directed) turning preferably in U shape is imparted to the flexural spring 6, wherein also other shapes are imaginable as they have been briefly mentioned at the beginning of the description.

In the present embodiment the cross web 6c is extended beyond the connecting points including the spring arms 6b in the direction of the clip branches and the actuating portions 4, respectively. Moreover, the cross web 6c has on its longitudinal side facing the spring legs 6b a rounding or pitch circle shaped notch between the connecting points including the spring legs 6b. Both structural measures serve for reducing notch effects and for avoiding a spring fracture at least within the scope of the provided deforming movements. In addition, hereby a spring-elastic characteristic which is then added to the spring arms 6b in a serial manner can be imparted to the cross web 6c. It is mentioned in this context that the flexural spring or its spring arms 6b forming the turning can be basically lengthened even further in the direction of the clamping bars 2a, as long as a sufficient distance for the flexural spring 6, i.e. the turning, is provided between the clip branches 1. It is decisive that a spring fracture is counteracted by the obtained length of the spring arms during normal use without extremely extending the outer dimensions of the clip and while maintaining a sufficient clamping force.

The cross web 6c also exhibits a further function, however.

As explained in the foregoing, the cross web 6c is lengthened beyond the connecting points including the spring arms 6b in the direction of the two actuating portions 4 and thus forms two stop projections or pins. The length of the cross web 6c is dimensioned so that upon actuation of the clip, i.e. when the actuating portions 4 are (elastically) compressed for opening/spreading the clamping portions 2, at a predetermined degree of opening the actuating bars 4a abut against the cross web 6c which thus blocks further compressing movement. In this way the flexural spring arrangement is prevented from being overstretched and damaged.

The pivoting motion of the two clip branches (in the opening and closing directions) is furthermore defined by the shape and material thickness of at least the two spring arms 6b and the cross web 6c as well as preferably also of the two actuating bars 4a especially when they are provided to be spring-elastic over their entire or only partial bar length and thus appropriately belong to the flexural spring arrangement 6. When the spring arms 6b and the actuating bars 4a have a comparatively elastic design and the cross web 6c has a rigid design (as provided in the present example), a substantially orbital pivoting movement of the two clip branches 1 along a pivoting link is resulting. That is to say, in this case the clip does not only have one pivot point but several pivot points corresponding to each other, whereby an individual movement mechanism is resulting.

When, on the other hand, the spring arms 6b forming the turning are designed to be relatively elastic and the actuating bars 4a are designed to be more rigid compared thereto, the pivot point is displaced to the area of the cross web 6c. In this case the pivoting movement of the two clip branches 1 is rather hinge-shaped around a pivot point.

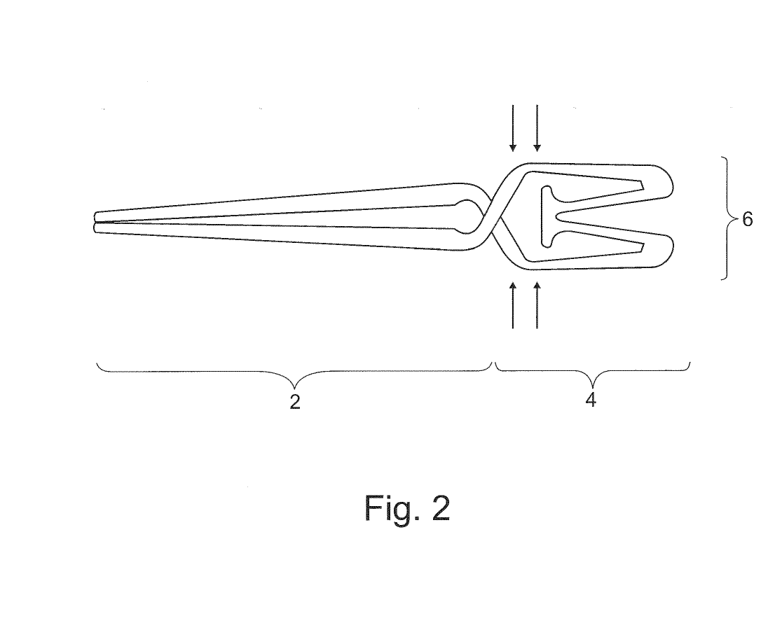

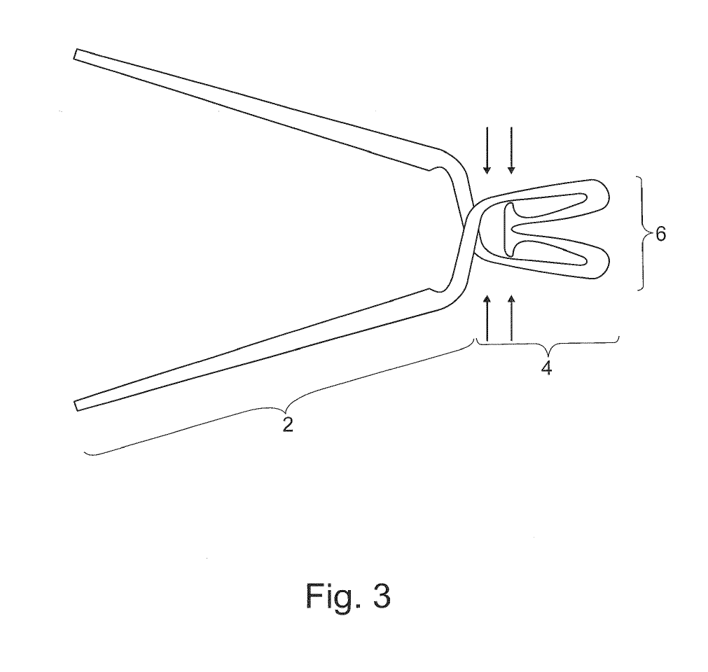

FIGS. 2 and 3 illustrate the clip according to the invention completely assembled in closed and open position. Consequently, the clip is provided according to the type of scissors so that the two intermediate pieces 2b are loosely intersecting so that compressing of the actuating bars 4a causes the clamping bars 2a to spread. The spring arrangement 1 is connected so-to-speak seamlessly, i.e. in extension of the actuating bars 4a, to the clip branches 1. This results in a continuous layout which is especially suited for manufacture of the clip in one piece. Therefore, the clip according to the invention is prepared as a sheet member of one single material, for example by punching or laser-cutting, in the form shown in FIG. 1 and is subsequently hardened at least in the areas provided as flexural springs so as to obtain particular spring elasticity. Alternatively, it is also possible to manufacture the spring arrangement at least consisting of the spring arms 6b forming the turning (including the cross web 6c) or together with the actuating bars 4a separately from the clamping portions 2 of a spring steel (by cutting, punching or laser-cutting) and to then connect it to the clamping portions 2, for example by hard-soldering.

Summing up, a surgical clip is disclosed comprising two clip branches 1 each consisting of a preferably straightly extending (bar-shaped) clamping portion 2 and an actuating portion 4 as well as comprising a flexural spring arrangement 6 by which the two clip branches are coupled in one piece. According to the invention, the flexural spring arrangement 6 is arranged and shaped at least in partial areas (centrally) so that it bulges between the clip branches 1 along the actuating portions 4 in the direction of the clamping portions 2. Irrespective thereof, the flexural spring arrangement 6 is composed by a number of serially coupled individual spring arms. The clamping portion is preferably defined as the axial portion of a branch which, when the pertinent (connected) actuating portion moves toward the opposite actuating portion, moves away from the opposite clamping portion. The pivot point of the two branches is defined by the spring arms of the flexural spring arrangement connecting the two clip branches and preferably additionally by the actuating portions which are spring-elastic at least over a partial length or else completely.

1-14. (canceled)

15. A surgical clip comprising two clip branches each consisting of a clamping portion and an actuating portion as well as comprising a flexural spring arrangement by which the two clip branches are coupled to each other in one piece, wherein the flexural spring arrangement bulges at least in partial areas between the clip branches along the actuating portions toward the clamping portions and in the area of said bulge forms at least two spring arms which are connected in one piece to the axial free ends of the actuating portions of the two clip branches, wherein the flexural spring arrangement includes a connecting web extending toward the two actuating portions for interconnecting the at least two spring arms at their respective one end facing the clamping portions, wherein the connecting web forms a stop for the actuating portions to delimit the actuating path thereof.

16. The surgical clip according to claim 15, wherein the flexural spring arrangement is composed of a plurality of serially coupled spring arms.

17. The surgical clip according to claim 15, wherein the flexural spring arrangement forms a turning preferably having a U, C, V or W shaped outline in its bulging partial area.

18. The surgical clip according to claim 15, wherein the connecting web projects beyond the connecting points including the spring arms in the direction of the two actuating portions of the clip branches so as to form an actuation limit stop.

19. The surgical clip according to claim 15, wherein the clip has a straight branch design, with each of the two opposing clamping portions thereof exhibiting a straightly extending clamping bar preferably equipped with a toothing or corrugation.

20. The surgical clip according to claim 19, wherein the two rear ends of the two clamping bars facing each of the actuating portions are lengthened into two intermediate pieces which in the construction position of the clip extend away from each other at an obtuse angle with the respective clamping bars.

21. The surgical clip according to claim 20, wherein the two opposed intermediate pieces are converted, at an obtuse angle, into the two actuating portions which approach each other in the direction of their rear free ends.

22. The surgical clip according to claim 15, wherein each of the two actuating portions forms an actuating bar which is spring-elastic over its entire or partial length so as to serve as further serially arranged spring arms of the flexural spring arrangement.

23. The surgical clip according to claim 15, wherein the clip is a sheet component which is preferably manufactured by punching or laser-cutting.

24. The surgical clip according to claim 15, wherein the flexural spring arrangement is made of spring steel separately from the clip branches and is connected to the two clip branches in one piece preferably by soldering or welding.

25. The surgical clip according to claim 15, wherein the clip is manufactured in one piece of one single material.

26. The surgical clip according to claim 15, wherein the clip branches extend while being loosely intersected such that the clamping portions and the actuating portions are functionally arranged on the one common side of the flexural spring arrangement, wherein movement of the actuating portions toward each other causes the clamping portions to move apart.
